(19) United States
(12) Patent Application Publication
(10) Pub. No.: US 2024/0354649 A1
(43) Pub. Date: Oct. 24, 2024

(54) PREDICTING HEALTH CONDITIONS BASED ON BIOPSY IMAGES USING MACHINE LEARNING

(71) Applicant: CancerRisk AI Inc., Greenbrae, CA (US)

(72) Inventors: Indra John Heckenbach, Santa Cruz, CA (US); Morten Scheibye-Knudsen, Hellerup (DK); Mark Joseph Powell, Greenbrae, CA (US)

(21) Appl. No.: 18/640,698

(22) Filed: Apr. 19, 2024

Related U.S. Application Data
(60) Provisional application No. 63/497,273, filed on Apr. 20, 2023.

(51) Int. Cl. G06N 20/00; G16H 50/30

(52) U.S. Cl. CPC G06N 20/00; G16H 50/30

(57) ABSTRACT

Techniques for predicting health conditions of patients using machine learning are described. Nuclei of cells from an image of a biological sample of the patient are identified. Cell type regions are identified that include one or more cells that are shown in the image. Cell types of the identified nuclei are identified based in part on locations of the nuclei relative to some or all of the identified cell type regions. Sets of scores for the identified nuclei are predicted using prediction models associated with different cell types. A set of aggregate scores for each of the cell types is generated using the sets of scores. A cancer risk or health outcome is estimated by applying at least some of the set of aggregate scores to a risk model.

### CROSS-REFERENCE TO RELATED APPLICATIONS

This application claims the benefit of U.S. Provisional Application No. 63/497,273, filed Apr. 20, 2023, which is incorporated by reference in its entirety.

### FIELD OF THE INVENTION

This disclosure relates generally to predicting cancer risk, and more specifically to predicting future cancer risk based on biopsy images using machine learning.

### BACKGROUND

Despite recent medical advances, cancer continues to take a devastating toll on the health of Americans, with 2 million new cases each year resulting in over 600,000 deaths. Although there have been advances in treatment, an important factor contributing to survival rate is early detection when the cancer is more likely to be localized and more easily curable. Current techniques for predicting risk of cancers such as breast cancer are primarily based on established risk factors such as age, parity, and family history. However, models based on these factors (such as the Gail model for breast cancer) have significant limitations in their predictive capabilities and are frequently unreliable.

### SUMMARY

In accordance with one or more aspects of the disclosure, techniques for predicting health conditions (e.g., cancer risks, health outcomes) based on biopsy images using machine learning are described. A health prediction computing system may predict one or more health conditions based on one or more images of a biological sample (e.g., tissue or biofluid) of a patient. The biological sample may be obtained via a biopsy of the patient. The health prediction computing system uses one or more prediction models to predict one or more sets of scores for a given image of a biological sample. In some embodiments, each set, of the sets of scores, is associated with a different cell type (e.g., stroma, fat, etc.). The aggregated scores may be applied to a risk model to determine a health condition of the patient. In some embodiments, the risk model may also use external factor data (e.g., age, gender, medical history, etc.) associated with the patient to determine the health condition. The health prediction computing system may directly or indirectly provide the health condition to a user.

In some aspects, the techniques described herein relate to a method, performed at a computer system including a processor and a non-transitory computer readable medium, including: identifying nuclei of cells from an image of a biological sample of a patient; identifying cell type regions that include one or more cells that are shown in the image; identifying cell types of the identified nuclei, based in part on locations of the nuclei relative to some or all of the identified cell type regions; predicting sets of scores for the identified nuclei using prediction models associated with different cell types, and that the sets of scores includes a respective set of scores for each of the different cell types; generating a set of aggregate scores for each of the cell types using the sets of scores, wherein the set of aggregate scores includes an aggregate score for each of the different cell types; and estimating a cancer risk for the patient by applying at least some of the set of aggregate scores to a risk model.

In some aspects, the techniques described herein relate to a method, performed at a computer system including a processor and a non-transitory computer readable medium, including: identifying nuclei of cells from an image of a biological sample of a patient; identifying a cell type or tissue type, which is to say it could be identifying types of individual cells or cells of a tissue that may include heterogenous cells of each nucleus of the identified nuclei; determining morphology of the identified nuclei; predicting sets of scores for the identified nuclei using prediction models associated with different cell types, and that the sets of scores includes a respective set of scores for each of the different cell types; generating a set of aggregate scores for each of the cell types using the sets of scores, wherein the set of aggregate scores includes an aggregate score for each of the different cell types; and estimating a health outcome for the patient by applying at least some of the set of aggregate scores to a risk model.

In some aspects, the techniques described herein relate to a non-transitory computer-readable storage medium including stored instructions, the instructions when executed by a processor of a device, causing the device to: identify nuclei of cells from an image of a biological sample of a patient; identify cell type regions that include one or more cells that are shown in the image; identify cell types of the identified nuclei, based in part on locations of the nuclei relative to some or all of the identified cell type regions; predict sets of scores for the identified nuclei using prediction models associated with different cell types, and that the sets of scores includes a respective set of scores for each of the different cell types; generate a set of aggregate scores for each of the cell types using the sets of scores, wherein the set of aggregate scores includes an aggregate score for each of the different cell types; and estimate cancer risks for the patient by applying at least some of the set of aggregate scores to a risk model.

### BRIEF DESCRIPTION OF THE DRAWINGS

FIG. 1 illustrates an example environment for a stress performance training system, in accordance with one or more embodiments.

FIG. 2 is a flowchart illustrating a process for assessing cancer risk from senescence, according to one or more embodiments.

FIG. 3 is a flowchart illustrating a process for assessing cancer risk based on cell type proximity, according to one or more embodiments.

FIG. 4 is a flowchart illustrating a process for assessing cancer risk based on spatial distribution(e.g., clustering), according to one or more embodiments.

FIG. 5 is a flowchart illustrating a process for predicting cancer risk from direct training on healthy tissue, according to one or more embodiments.

FIG. 6 is a flowchart illustrating a process for predicting cancer risk from direct training on healthy tissue by morphology, according to one or more embodiments.

FIG. 7 is a flowchart illustrating a process for predicting cancer risk from developing senescence phenotypes, according to one or more embodiments.

FIG. 8 is a flowchart illustrating a process for predicting cancer risk from multiple cell state phenotype, according to one or more embodiments.

FIG. 9 is a flowchart illustrating a process for predicting a process for predicting inflammation and health outcomes from senescence, according to one or more embodiments.

FIG. 10 is a flowchart illustrating a process for predicting a health condition using one or more prediction models, in accordance with one or more embodiments.

The figures depict various embodiments for purposes of illustration only. One skilled in the art will readily recognize from the following discussion that alternative embodiments of the structures and methods illustrated herein may be employed without departing from the principles described herein.

### DETAILED DESCRIPTION

A health prediction computing system predicts a health condition (e.g., risk of cancer or health outcome) based on one or more images of a biological sample (e.g., tissue or biofluid) of a patient. The biological sample may be obtained via a biopsy from the patient. The health prediction computing system may be applied for estimation/assessment of risk of breast cancer, prostate cancer, skin cancer, various types of gastrointestinal cancer, blood cancer, lung cancer, or other types of cancers. Use of the health prediction computing system to predict health risk may result in significant improvements in early detection leading to the ability to more successfully treat cancer while also reducing the need for repeat biopsies and other invasive screening procedures.

An example embodiment enables the prediction of future risk of cancer from an image of tissue. The image is from tissue taken at a biopsy which did not find cancer and uses its standard staining and/or some other technique to identify nuclei (e.g., 4′, 6-diamidino-2-phenylindole fluorescent stain also referred to as DAPI). Segmentation is applied to identify different tissue cell types (such as epithelium, fat, and stroma in breast tissue) and the nuclei of those specific cells. The location of each nucleus in proximity to other tissue types is determined. A senescence score is generated for each nucleus based on trained senescence models applied to the morphology of each nucleus. Senescence models were generated from cells that are exposed to radiation and drugs known to cause senescence or passaged without treatment until reaching replicative senescence, with one or more prediction models trained by mapping images of nuclear morphology to the senescent state resulting from each senescence-generating method. Scores for each nucleus of each tissue type for each senescence model are combined to generate aggregate scores. In some embodiments, aggregate scores are generated based on normalized scores and may reflect changes in the distribution of scores per model per tissue per patient, which can be useful in evaluating patients independent of a specific cohort. Future cancer risk is estimated by comparing each of these aggregate scores to information (e.g., a database of scored samples or derived summary data) in a risk model. In some embodiments, the estimated cancer risk may be based in part on results from multiple senescence models in multiple component tissues and external risk factors (e.g., age, gender, etc.).

In an example implementation, a machine-learned senescence-scoring algorithm evaluates one or more core biopsy samples from an organ-at-risk. Risk scores are produced from some or all of the organ's different resident cell components. These cell-specific scores may enhance and better personalize cancer risk assessment, increasing early detection in those found to be at high risk and reducing anxiety and costs by decreasing unnecessary screening in those found to be at lower risk. At least some of the described techniques are based in part on detection of senescent cells. Senescent cells are cells which no longer divide but remain metabolically active and have complex associations with tumor development. Senescent cells sometimes result in a tumor protective environment, while at other times promote tumor development by emitting a variety of factors known as the senescence associated secretory phenotype (SASP), with senescent cells secreting high levels of inflammatory cytokines, growth factors, and proteases. These effects vary from tissue to tissue and may relate to spatial associations of the senescent cells with specific cell types in the target tissue.

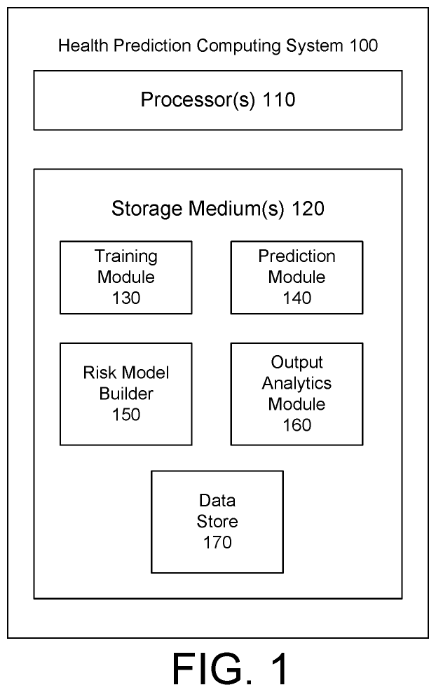

FIG. 1 illustrates an example embodiment of a health prediction computing system 100 that may be employed to implement the techniques described herein. The health prediction computing system 100 includes one or more processors 110 and one or more non-transitory computer-readable storage mediums 120 that store instructions executable by the one or more processors 110 for carrying out the functions described herein. The instructions may be logically grouped into various functional modules. The functional module may include a training module 130, a prediction module 140, a risk model builder 150, and an output analytics module 160. In various embodiments, the storage medium may store additional modules for carrying out other functions described herein. Furthermore, the storage medium may store other data besides executable instructions (e.g., images) in a data store 170. Some embodiments of the health prediction computing system 100 have different components than those described here. Similarly, in some cases, functions can be distributed among the components in a different manner than is described here.

The health prediction computing system 100 may be implemented as a standalone computing system, as a networked computing system (e.g., using a client/server architecture), as a cloud computing or other distributed computing architecture, or a combination thereof.

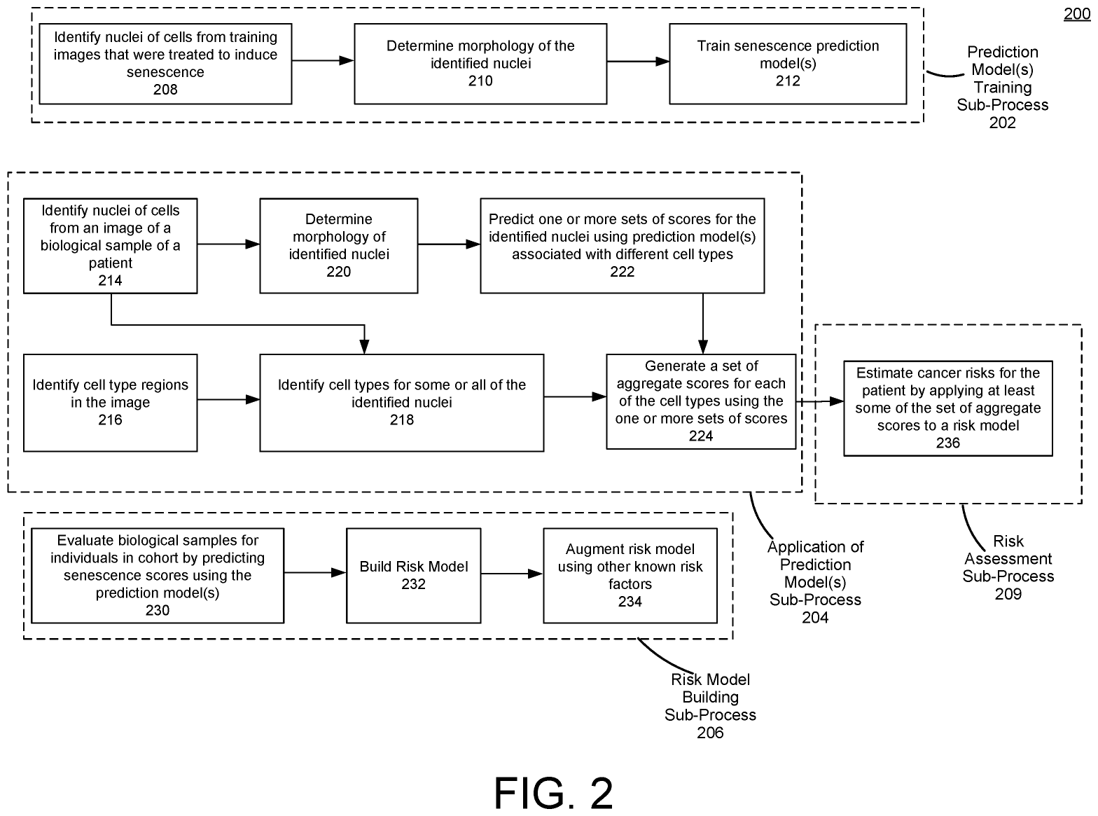

The training module 130 trains machine learning models used by the health prediction computing system 100. For example, the training module 130 may train one or more prediction models, and in some embodiments one or more risk models, as described below with regard to FIGS. 2-10. The health prediction computing system 100 may use machine learning models to perform functionalities described herein. For example, the training module 130 may train one or more prediction models that map cell images (or features derived therefrom) to cancer risk assessments and/or other related predictions. Example machine learning models include regression models, support vector machines, naïve bayes, decision trees, k nearest neighbors, random forest, boosting algorithms, k-means, and hierarchical clustering. The machine learning models may also include neural networks, such as perceptrons, multilayer perceptrons, convolutional neural networks, recurrent neural networks, sequence-to-sequence models, generative adversarial networks, or transformers.

Each machine learning model includes a set of parameters. A set of parameters for a machine learning model are parameters that the machine learning model uses to process an input. For example, a set of parameters for a linear regression model may include weights that are applied to each input variable in the linear combination that comprises the linear regression model. Similarly, the set of parameters for a neural network may include weights and biases that are applied at each neuron in the neural network. The training module 130 generates the set of parameters for a machine learning model by “training” the machine learning model. Once trained, the machine learning model uses the set of parameters to transform inputs into outputs.

The training module 130 trains a machine learning model based on a set of training examples. Each training example includes input data to which the machine learning model is applied to generate an output. For example, each training example may include one or more training images of tissue including nuclei (e.g., images of cells found in blood smears, images of nuclei of cells found in sputum), training images of nuclei where images have been normalized by size, color, background, or only nuclear morphology is visible, training images of cells in which at least some of the cells were treated to induce replicative senescence by serial passaging, training images of cells in which at least some of the cells were treated to induce senescence through drug treatment, training images of the cells in which at least some of the cells were treated to induce a stressed cell state (e.g., senescence, quiescence), training images of cells where at least some of the cells in the training images were treated to induce senescence, risk metrics, known risk factors (e.g., such as age, personal medical history, family medical history, Gail score, etc.), some other training example relevant to training a prediction model, some other training example relevant to training a risk model, or some combination thereof. In some cases, the training examples also include a label (e.g., an identifier for treated or untreated groups, a numerical value related to presence of stain or stained marker indicating senescence, a numeric value indicating likelihood of senescence or other numerical value that increases above a threshold indicating senescence state) which represents an expected output of the machine learning model. In these cases, the machine learning model is trained by comparing its output from input data of a training example to the label for the training example.

The training module 130 may apply an iterative process to train a machine learning model whereby the training module 130 trains the machine learning model on each of the set of training examples. To train a machine learning model based on a training example, the training module 130 applies the machine learning model to the input data in the training example to generate an output. The training module 130 scores the output from the machine learning model using a loss function. A loss function is a function that generates a score for the output of the machine learning model such that the score is higher when the machine learning model performs poorly and lower when the machine learning model performs well. In cases where the training example includes a label, the loss function is also based on the label for the training example. Some example loss functions include the mean square error function, the mean absolute error, hinge loss function, and the cross-entropy loss function. The training module 130 updates the set of parameters for the machine learning model based on the score generated by the loss function. For example, the training module 130 may apply gradient descent to update the set of parameters.

In some embodiments, the training module 130 may update (e.g., re-train) the one or more prediction models based on information from the prediction module 140 and/or the output analytics module 160.

The prediction module 140 applies the one or more prediction models trained by the training module 130 to one or more input images to generate one or more predictions. For example, the one or more input images may be of a biological sample (e.g., tissue) of a patient. For example, following biopsy, tissue is prepared so nuclei are visible. Biopsy tissue may be stained with H&E (or other stains such as DAPI), and then imaged with a slide scanner or other microscope to produce a digitized image of tissue including nuclei that may be input into the prediction module 140.

The prediction module 140 is configured to identify nuclei of cells from the one or more received images. In some embodiments, the prediction module 140 applies segmentation to the one or more images to identify nuclei of cells present in the images. This segmentation may be performed using techniques such as a U-Net model trained on different cell types (e.g., adipose, stroma, epithelium, etc.) to identify nuclei of cells in a given image of a biological sample from a patient.

The prediction module 140 is configured to identify cell type regions from the one or more received images. Cell type regions are regions of cells or tissue of a common type or of a predominantly common type. For example, tissues like adipose, stroma, and epithelium, are heterogenous, but often include a primary cell type. In some embodiments, the prediction module 140 applies segmentation to the one or more images to identify different cell type regions present in the images. This segmentation may be performed using techniques such as a U-Net model trained on adipose, stroma, and epithelium to predict cell type regions of each image.

The prediction module 140 identifies a cell type for each nucleus based on detected location of the nucleus relative to the segmented areas of different cells or tissue types identified in the input images. For each nucleus identified in the input images, the prediction module determines whether the location of the nucleus is associated with an identified cell or tissue type based on its location. For example, the prediction module maps the location of each nucleus to the detected segmentation regions to determine overlap.

The prediction module 140 may predict one or more sets of scores for the identified nuclei using one or more prediction models. In some embodiments, each set of the one or more scores is associated with different cell types. Most cells are only one cell type region (e.g., stroma, fat, etc.), and there may be multiple prediction models for each type, so each nucleus may have multiple scores like 0.1, 0.15, 0.18, etc. (an ensemble), but still only be of one cell type region. Additionally, multiple prediction models may be used such as several IR-induced models, several RS models, several based on drug treatment, so each nucleus would have several scores from each class of model: 0.1, 0.15, 0.18 for IR models, 0.2, 0.25, 0.8 for RS models, 0.9, 0.91, 0.92 for doxorubicin-induced models. Each set of models (e.g. IR models) may predict on nuclei of all tissue types or in some embodiments be specialized by tissue type. In some embodiments there may be one score per nuclei per senescence-induction model like IR and each nucleus is of one cell/tissue type.

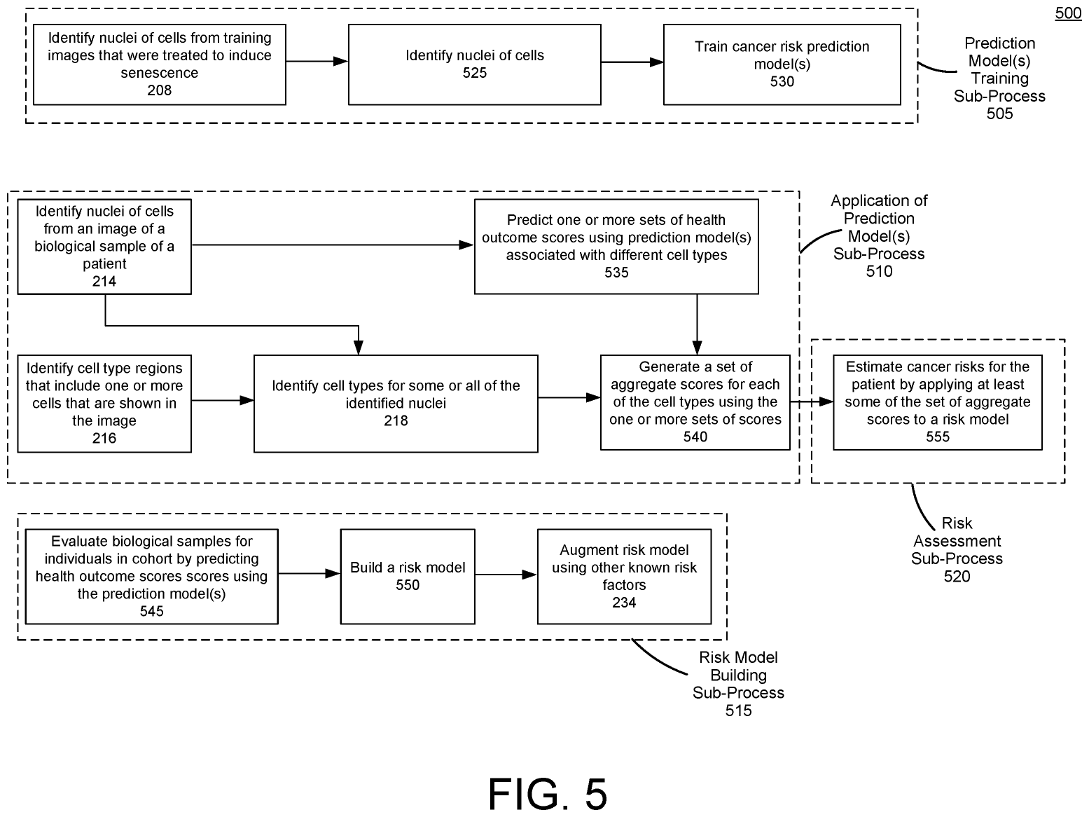

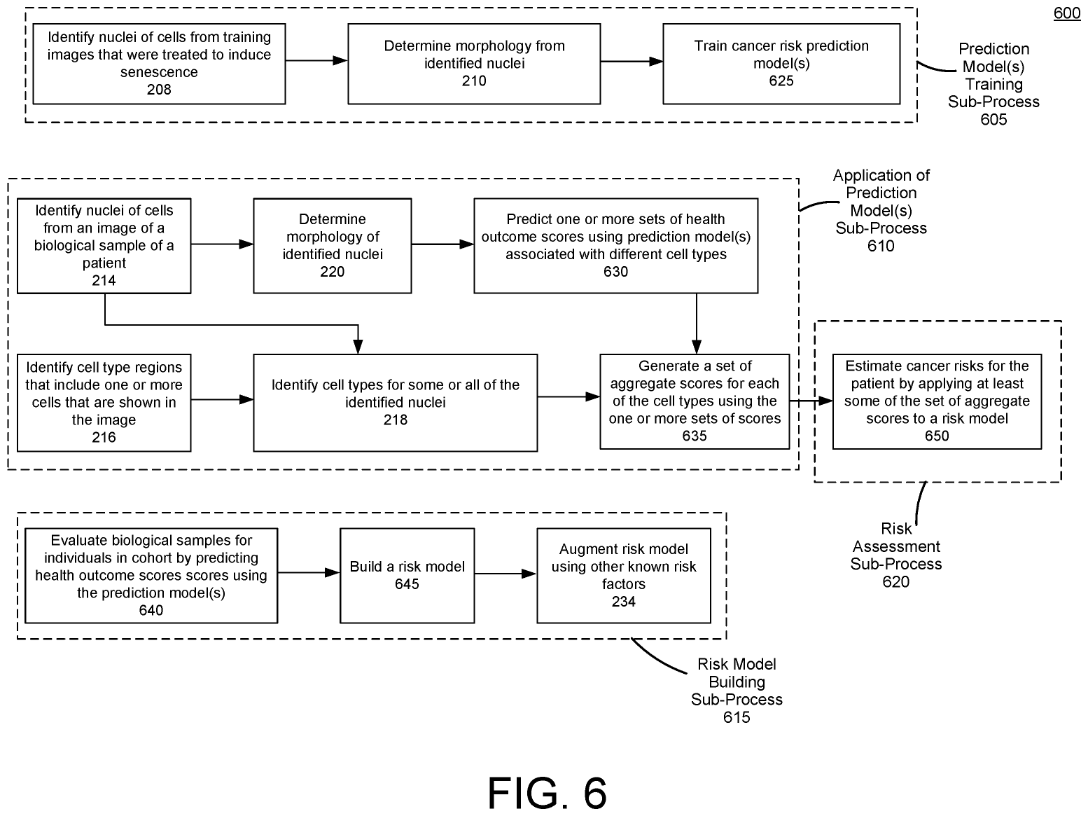

In some embodiments (e.g., FIGS. 2-4, 7, and 8), the scores predict senescence per nuclei for each trained model. Here, the trained senescence model from the training stage described above is applied to the segmented images of the nuclear morphology generated from the biopsy image. For each nucleus, a senescence score is generated by the trained model. In some embodiments (e.g., FIGS. 5 and 6), the scores are health outcome scores.

The prediction module 140 may generate aggregate scores for a tissue sample by combining scores for all nuclei per tissue/cell type for each model (if multiple models are used). In this step, all scores for each nucleus per tissue or cell type per patient are combined to generate aggregate scores per tissue or cell type per patient. Scores may be combined using a combining function such as mean, median, or normalized to produce an overall score based on the distribution of nuclei scores per model per tissue per individual. The combined score thus provides an overall assessment of the level of senescence in each tissue type of a patient. For example, in breast tissue different combined scores may be generated for tissue types such as the fat, stromal, epithelial, or other. Continuing with the example, given a first number (e.g., 1000) of scores for stroma cells, a second number. (e.g., 800) scores for fat cells, and a third number (e.g., 900) for epithelial cells, the prediction module 140 may aggregate (e.g., take mean, or normalize by subtracting the median value and dividing by the range between the first and third quartile) the first number of scores to form a first aggregated score, aggregate the second number of scores to form a second aggregated score, and aggregate the third number of scores to form a third aggregated score. In this manner, the prediction module 140 may reduce a large number of scores to a single score for each cell type. Note in some embodiments, the aggregated scores may be stratified in some manner (e.g., by spatial distribution of nuclei, distance between cell types, etc.).

The risk model builder 150 generates a risk model using the one or more prediction models that are used by the prediction module 140. The risk model builder 150 instructs the prediction module 140 to obtain a set of aggregate scores (e.g., a senescence score or a health outcome score) for each cell type. The one or more risk training cohorts may include patients with differing risk training data. Each patient in the one or more risk training cohorts is associated with a respective set of risk training data. Risk training data includes a risk training image of a biological sample and risk metrics (a percentage likelihood of having cancer, a risk category of having cancer such as low, medium, high, etc.) or health outcomes (e.g., disease prognosis or other outcomes related to immune system function, inflammation, specific diseases such as diabetes and cardiovascular disease, etc.), and may also include information about the patient like, e.g., age, gender, race, medical history (e.g., having some type of cancer, level of cancer progression), etc. The risk training images may comprise cell images from a single tissue type (e.g., breast tissue, prostate tissue, etc.) or may include images from a variety of different tissue types. In some embodiments, some or all of the actions of the risk model builder 150 are automated and/or machine learned. In some embodiments, some of the actions of the risk model builder 150 may be performed by a human (e.g., selecting thresholds based on cohort results, such as all scores above 0.6 for IR in adipose tissue and below 0.8 for Doxo in stromal tissue have risk of 276% above the base line, or other simpler or more complex tables of aggregate score associated risk).

The risk model builder 150 builds a risk model. In some embodiments, the risk model builder 150 builds a risk model that maps the set of scores for each of the individuals in the training cohort to a risk metric (e.g., a percentage likelihood of having cancer) or one or more health outcomes using the risk training data. In some embodiments, the risk model is a machine learned model, and the risk model builder 150 may use the training module 130 to train the risk model. For example, the training module 130 may train the risk model using training examples for individuals in the risk training cohort.

In some embodiments, the risk model builder 150 may augment the risk model using other known risk factors. For example, the risk model builder 150 may augment risk prediction of the risk model to account for other known risk factors (e.g., such as age, personal medical history, family medical history, Gail score, etc.) from the risk training data. In some embodiments, the risk model builder may apply one or more ensemble learning methods to optimize risk prediction by the risk model with addition of other known risk factors.

The output analytics module 160 performs risk assessment using the aggregated scores and the risk model. For example, the output analytics module 160 may estimate cancer risks and/or health outcomes for the patient by applying at least some of the set of aggregate scores to the risk model.

The output analytics module 160 generates various outputs based on the predicted results. For example, the output analytics module 160 may generate various user interface elements such as scores, charts, graphs, or other visual indicators related to the estimated cancer risks and/or health outcomes. The output analytics module 160 may furthermore generate recommendations relating to treatment methods, diagnosis information, prognosis information, or other information that may be derived from the risk predictions.

The data store 170 stores data used by the health prediction computing system 100. For example, the data store 170 stores images of biological samples, training images of biological samples, scores generated by prediction model(s), aggregated scores, risk training data (e.g., risk training images, risk metrics, known risk factors for patients, etc.), information used in the risk model, estimated health conditions (e.g., estimated health outcome, estimated cancer risk) some other information for use by the health prediction computing system 100. The data store 170 also stores trained machine learning models (e.g., the one or more prediction models, and in some embodiments, the risk model) trained by the training module 130. The data store 170 uses computer-readable media of the storage mediums 120 to store data.

FIGS. 2-10 are flowcharts illustrating various example processes associated with predicting cancer or other health-related outcomes based on input images of cells. FIG. 2 is a flowchart illustrating a process 200 for assessing cancer risk from senescence, according to one or more embodiments. The process 200 includes several sub-processes including a prediction model(s) training sub-process 202, an application of prediction model(s) sub-process 204, a risk model building sub-process 206, and a risk assessment sub-process 209 (that uses the prediction model(s) and the risk model). The process and/or sub-processes shown in FIG. 2 may be performed by components (e.g., the training module 130, the prediction module 140, the risk model builder 150, and the output analytics module 160). Other entities may perform some of the steps in FIG. 2 in other embodiments. Embodiments may include different and/or additional steps, or perform the steps in different orders.

Note that the process 200 is directed toward assessing cancer risk. The type of cancer risk being checked for is determined on how the models used by the process 200 are trained. For example, they may be trained to assess risk of breast cancer, prostate cancer, some other cancer, or some combination thereof. In other embodiments, the process 200 may use prediction models that are not trained for cancer (but a risk model that was trained using a cancer cohort).

### (1) Prediction Model(s) Training Sub-Process 202

The training module 130 receives training images. The images may be of cells (or parts of cells containing nuclei). The training images may include images taken from different individuals with differing physical characteristics such as varying age, gender, race, medical histories, etc. The training images may be based on images of cells extracted from human or other species such as mouse. The training images may comprise cell images from a single tissue type (e.g., breast tissue, prostate tissue, etc.) or may include images from a variety of different tissue types. At least some of the nuclei in the training images may have been treated to induce senescence. For example, human fibroblasts or other cell types from humans or other species such as mouse are passaged for an extended period of time to induce replicative senescence, exposed to ionizing radiation to induce DNA damage and cellular senescence, or treated with various compounds such as Antimycin A, Doxorubicin, ATV/r to induce senescence through diverse mechanisms including but not limited to proteotoxic stress, mitochondrial dysfunction, oxidation, or DNA damage. Both senescence induced cells and normal control cells are imaged, optionally stained with markers to identify nuclei such as DAPI, H&E, LMNB1, using brightfield, phase contrast, confocal, or other microscopes. The microscope output may be digitized to form the training images.

As part of the prediction model(s) training sub-process 202, the training module 130 may identify 208 nuclei of cells from the training images, where the cells in the training images were treated to induce senescence. In some embodiments, the training module 130 performs one or more segmentation techniques to identify nuclei in the training images. Segmentation techniques applied to identify individual nuclei may include, for example, traditional imaging methods such as gaussian blur and thresholding or application of deep neural networks. Deep neural networks may be applied using pretrained models that were previously trained on diverse samples or as custom segmentation models, where nuclei are annotated on one set of slides, used to train a deep model such as U-Net, and then used to predict nuclei across the set of image samples.

The training module 130 may determine 210 morphology of some or all of the identified nuclei. For example, the training module 130 may standardize images of individual nuclei by converting the longer of their dimensions to a standard size (with or without first rotating to position their longer dimension on one axis), and removing the background detail and foreground, leaving only nuclei morphology images.

The training module 130 may train 212 one or more prediction models (e.g., senescence prediction models). The one or more prediction models are machine learning models. The training module 130 may train a prediction model to map determined morphology (i.e., the nuclei morphology images) to a corresponding senescent state using classifiers which may generate an output state (e.g., “sen” or “not sen”) or may also generate numerical scores (such as softmax). In some embodiments, the training module 130 trains a single prediction model for predicting senescence in a plurality of different cell types. In some embodiments, the training module 130 trains different prediction models for predicting senescence for different cell types. For example, one prediction model trained to predict senescence in adipose cells, another prediction model trained to predict senescence in stroma cells, etc. In the training stage, the training module 130 may use multiple training methods to generate a set of different senescence predictor models. For example, a set of nuclei morphology images (e.g., produced in step 210) may be used as the data to train a deep neural network to map nuclear morphology images to corresponding labeled senescence state outputs. Prior to the output function, the label may comprise a numeric value indicating likelihood of senescence or other numerical value that increases above a threshold indicating senescence state, such as the softmax function with one-hot output for senescent or non-senescent output states. The deep neural network may be a custom network with only a few layers or a large model such as Xception. The prediction model may utilize an ensemble of deep neural networks, each of which is trained independently, where their output scores or states are aggregated to produce an output senescence score (e.g., a mean of the numerical scores of each member of the ensemble).

### (2) Application of Prediction Model(s) Sub-Process 204

The prediction module 140 receives one or more images of a biological sample (e.g., tissue) of a patient. For example, following biopsy, tissue is prepared so nuclei are visible. Biopsy tissue may be stained with H&E, and then imaged with a slide scanner or other microscope to produce a digitized image of tissue including nuclei.

The prediction module 140 may identify 214 nuclei of cells from the one or more images of the biological sample. For example, the prediction module 140 may apply one or more segmentation techniques to the images to identify nuclei of some or all of cells present in the images. The one or more segmentation techniques may be performed using techniques such as a U-Net model trained on different cell types (e.g., adipose, stroma, epithelium, etc.) to identify nuclei in the one or more images.

The prediction module 140 may identify 216 cell type regions in the one or more images. For example, the prediction module 140 may apply one or more segmentation techniques to the one or more images to identify different cell type regions present in the one or more images. The one or more segmentation techniques may be performed using techniques such as a U-Net model trained with different cell types to predict cell type regions of each image. Note step 216 and step 214 may be performed in parallel with each other.

The prediction module 140 may identify 218 a cell type of some or all of the identified nuclei. For example, for a given nucleus, of the identified nuclei, the prediction module 140 may identify the cell type for that nucleus based in part on a location of the nucleus relative to some or all of the identified cell type regions. In some embodiments, the prediction module 140 may identify 218 a cell type of each nucleus, of the identified nuclei, based in part on a location of the nucleus relative to some or all of the identified cell type regions. In some embodiments, the prediction module 140 may map the location of each nucleus to the detected cell type regions to determine overlap.

The prediction module 140 may determine 220 morphology and/or other morphological properties of the identified nuclei. The prediction module 140 may perform the step 220 in substantially the same or similar manner as described above with respect to step 210.

The prediction module 140 may predict 222 one or more sets of scores for the identified nuclei using the one or more prediction models that are associated with different cell types. The prediction module 140 may predict senescence scores per nuclei for each trained prediction model. For example, the one or more trained prediction models from the prediction model(s) training sub-process 202 described above are applied to the segmented images of the nuclear morphology generated from the image of the biological sample. For some or all of the identified nuclei, a senescence score is generated by the one or more prediction models. Note that the one or more prediction models generate a score for each cell type. For example, in some embodiments, each of the one or more prediction models generates a score for a particular cell type. In other embodiments, a single prediction model may generate scores for multiple cell types. Accordingly, the one or more prediction models may generate a plurality of sets of scores for the identified nuclei (e.g., scores for nuclei from stroma cells, scores for nuclei from epithelial cells, etc.).

The prediction module 140 may generate 224 a set of aggregate scores for each of the cell types using the one or more sets of scores. In some embodiments, the prediction module 140 generates aggregate scores for the biological sample by combining scores for all nuclei per cell type for each prediction model (if multiple models are used). For example, all scores of the patient for each nucleus associated with a particular cell type may be combined to generate aggregate scores for that cell type. The prediction module 140 may combine the scores for a given cell type using a combining function such as mean, median, some other aggregating function that may utilize raw or normalized scores, or some combination thereof to obtain an aggregate score. Accordingly, the prediction module 140 generates a set of aggregated scores, where each aggregated score is associated with a different cell type (e.g., fat, stromal, epithelial, etc.). The set of aggregate scores may thus provide an assessment of levels of senescence in one or more cell types of a patient.

Note that while the application of prediction model(s) sub-process 204 described above may be a use case that generates a set of aggregate scores, some or all of the application of prediction model(s) sub-process 204 may be used as part of the risk model building sub-process 206 to build a risk model. For example, the prediction module 140 may use some or all of the application of prediction model(s) sub-process 204 to facilitate building of a risk model as described below.

### (3) Risk Model Building Sub-Process 206

In the risk model building sub-process 206, the risk model builder 150 evaluates 230 biological (e.g., tissue) samples for individuals in a risk training cohort by predicting senescence scores using the one or more predictor model(s). For example, the risk model builder 150 may instruct the prediction module 140, for each individual in a risk training cohort, to apply the application of prediction model(s) sub-process 204 to generate a set of aggregate scores (e.g., one score for each cell type).

The risk model builder 150 builds 232 a risk model. In some embodiments, the risk model builder 150 builds a risk model that maps the set of aggregated scores for each of the individuals in the training cohort to a risk metric. In this example, the risk metrics may include, e.g., a percentage likelihood of having cancer, a risk category of having cancer (e.g., low, medium, high), etc. For example, utilizing the predicted scores and observed case outcomes for patients in the training cohort, the risk model builder 150 can calculate risk or apply logistic regression models to estimate risk. In some embodiments, the risk model builder 150 may map the scores from the aggregated set of scores to risk, such as odds ratio by medians, quartiles, or higher precision groups or other methods to formulate associations between scores and risk. In some embodiments, the risk model is a machine learned model, and the risk model builder 150 may use the training module 130 to train the risk model. For example, the training module 130 may train the risk model using training data for individuals in the risk training cohort.

In some embodiments, the risk model builder 150 may augment 234 the risk model using other known risk factors. For example, the risk model builder 150 may augment risk prediction of the risk model to account for other known risk factors (e.g., such as age, personal medical history, family medical history, Gail score, etc.). In some embodiments, the risk model builder 150 may apply one or more ensemble learning methods to optimize risk prediction by the risk model with addition of other known risk factors.

### (4) Risk Assessment Sub-Process 209

In the risk assessment sub-process 209, the output analytics module 160 may estimate 236 cancer risks for the patient by applying at least some of the set of aggregate scores to the risk model (i.e., built as part of the risk model building sub-process 206). For example, the output analytics module 160 may estimate cancer risks by comparing the aggregate scores of particular cell types to information (e.g., database of scored samples and/or derived summary data) of the risk model. In some embodiments, the risk model may account for other known risk factors of the patient in estimating the cancer risks. The risk model may, e.g., combine one or more senescence scores with other risk factors. For example, high senescence scores for fat with IR model and low senescence scores for fat with AAD and high Gail score may significantly elevate risk, and can be translated to a specific risk estimate using the risk model developed on the risk training cohort.

The output analytics module 160 may provide the estimated cancer risks for presentation. The output analytics module 160 may provide the estimated cancer risks to a monitor of the health prediction computing system 100 for display and/or to a client device for display, where the client device is communicatively coupled to the health prediction computing system.

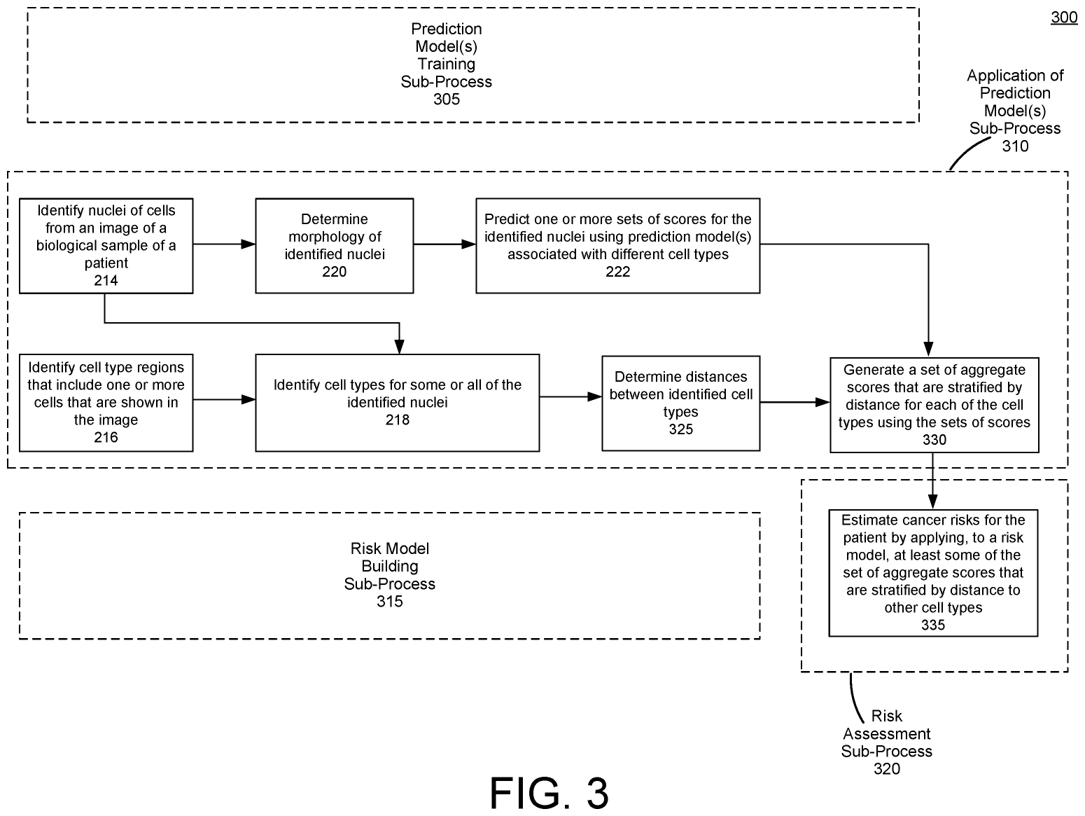

FIG. 3 is a flowchart illustrating a process 300 for assessing cancer risk based on cell type proximity, according to one or more embodiments. The process 300 includes several sub-processes including a prediction model(s) training sub-process 305, an application of prediction model(s) sub-process 310, a risk model building sub-process 315, and a risk assessment sub-process 320. The process and/or sub-processes shown in FIG. 3 may be performed by components of the health prediction computing system 100. Other entities may perform some of the steps in FIG. 3 in other embodiments. Embodiments may include different and/or additional steps, or perform the steps in different orders.

The prediction model(s) training sub-process 305 is substantially or similarly the same as the prediction model(s) training sub-process 202, and the risk model building sub-process 315 is substantially or similarly the same as the risk model building sub-process 206.

Furthermore, steps 214, 216, 218, 220, and 222 of the application of prediction model(s) sub-process 310 are substantially the same as the corresponding steps in the application of prediction model(s) sub-process 204. In the illustrated embodiment, the application of prediction model(s) sub-process 310, after step 218, has the prediction module 140 determine 325 distances between identified cell types. For example, for a given nucleus, the prediction module 140 calculates distances to other nuclei, while tracking the cell type of some or all of the identified nuclei. This can be done for each nucleus by recording the distance and type of every other nucleus. In some embodiments, the prediction module 140 may compute the distances in aggregate form such as generating summary results that capture distance to other cell types.

The prediction module 140 may generate 330 a set of aggregate scores that are stratified by distance for each of the cell types using the one or more sets of scores. The step 330 is similar to the step 224, except the set of aggregated scores are not stratified by distance. For example, the prediction module 140 may generate aggregate scores for a biological sample (e.g., tissue sample) by combining scores for all nuclei per cell type for each model, stratified by distance between cell types. For example, a set of aggregate scores may be generated using a particular model for mean epithelial cells' score by distance from fat cells: 1″ nearest spatial range=0.65, ₂″ nearest spatial range=0.67, 3^{rd} nearest spatial range=0.68. For each model and cell type, the prediction module 140 may calculate the aggregate senescence of other cell types, stratified or otherwise considering distance, or by means of a derivative metric such as half-life of scores' spatial trend or relative difference between near and far scores. The prediction module 140 may evaluate a mean senescence of nearby nuclei of other cell types. For example, when evaluating breast tissue for breast cancer risk, the prediction module 140 may generate an aggregate score representing a mean senescence of terminal duct lobular unit (TDLU) nuclei near adipose nuclei.

The risk assessment sub-process 320 is substantially similar to the risk assessment sub-process 209, except that step 236 is replaced with estimating 335 cancer risks for the patient by applying, to a risk model (i.e. the risk model produced by the risk model building sub-process 315), at least some of the set of aggregate scores that are stratified by distances to other cell types. For example, output analytics module 160 may estimate cancer risks by comparing the aggregate scores of particular cell types, stratified by distance to other types, to the risk model (e.g., may include a database of scored samples). This step may be similar to the analogous step 236, but employs a function that is affected by specific spatial patterns such as distance to other cell types.

The output analytics module 160 may provide the estimated cancer risks (e.g., output of step 335) for presentation as described above with reference to FIGS. 1 and 2.

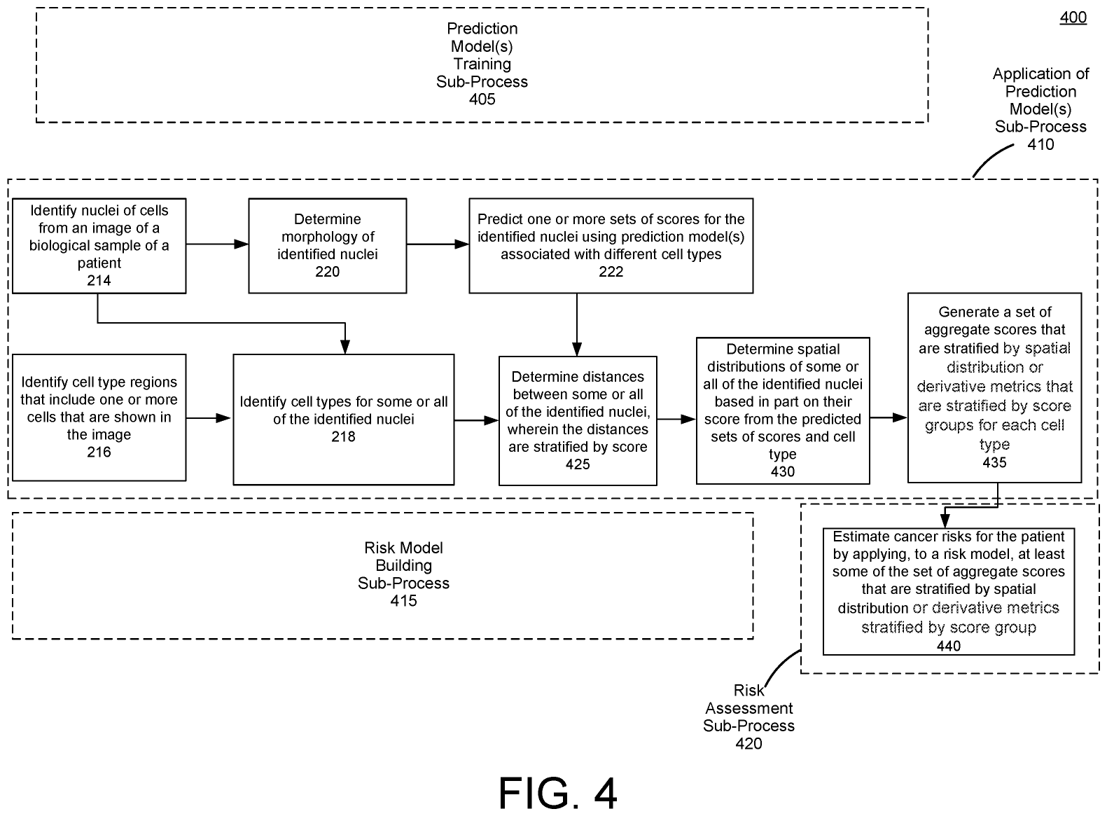

FIG. 4 is a flowchart illustrating a process 400 for assessing cancer risk based on spatial distribution(e.g., clustering), according to one or more embodiments. The process 400 includes several sub-processes including a prediction model(s) training sub-process 405, an application of prediction model(s) sub-process 410, a risk model building sub-process 415, and a risk assessment sub-process 420. The process and/or sub-processes shown in FIG. 4 may be performed by components of the health prediction computing system 100. Other entities may perform some of the steps in FIG. 4 in other embodiments. Embodiments may include different and/or additional steps, or perform the steps in different orders.

The prediction model(s) training sub-process 405 is substantially or similarly the same as the prediction model(s) training sub-process 202, and the risk model building sub-process 415 is substantially or similarly the same as the risk model building sub-process 206.

Furthermore, steps 214, 216, 218, 220, and 222 of the application of prediction model(s) sub-process 410 are substantially the same as the corresponding steps in the application of prediction model(s) sub-process 204. In the illustrated embodiment, the application of prediction model(s) sub-process 410, after step 218, determine 425 distances between some or all the identified nuclei, wherein the scores are stratified by distance to other score-stratified nuclei, from the predicted sets of scores. The distances between each pair of nuclei may be calculated, and nuclei may be organized into score groups, such as the highest 10 and lowest 10 percentiles of scores. All nuclei (of the same or different cell type) may be then aggregated according to their distance from the score group. In an example specific implementation, after identifying specific nuclei with percentile ranges of scores (low 0-10, 10-20, . . . , high 90-100), scores around those nuclei may be stratified by distance. So for example showing multiple nuclei relative to a central_nuclei_score_range, may include both generating distances between individual nuclei and stratifying by score group by generating mean scores for each stratified distance ring.

The prediction module 140 may determine 430 spatial distributions of some or all of the identified nuclei based in part on their score from the predicted sets of scores and cell type. For example, the prediction module 140 may identify spatial distributions (e.g., distance rings, clusters, etc.) of nuclei by cell type and score. The prediction module 140 may compute the calculated distances by cell type and score as described above, and then determine spatial distributions, stratified by cell type and score range. For example, clusters of high scoring adipose nuclei in near proximity or high rate of senescence score decline over distance from top scoring nuclei. In some embodiments, the prediction module 140 may use curve fitting techniques to determine spatial distributions of some or all of the identified nuclei based in part on their score from the predicted sets of scores and cell type.

The prediction module generates 435 a set of aggregate scores that are stratified for each of the cell types using the set of scores. The stratification may be by spatial distribution or derivative metrics that are stratified by score group. For example, the prediction module 140 may generate aggregate scores for the biological sample by combining scores for nuclei per cell type for each model, stratified by spatial distribution, relative to high scoring nuclei. An example set of aggregated scores stratified by spatial distribution may be score mean of nearest region is 0.66, score mean of 2^{nd} nearest region is 0.67, relative to the 90+ percentile scoring nuclei per cell type. In some embodiments, the prediction module 140 may determine the spatial distribution of scores for each cell type and between different cell types, such as clusters of high scoring adipose nuclei or a weakly declining distribution of senescence in stroma, near other cell types. The spatial distribution may be quantified using a derivative metric such as half-life of scores' spatial decay, relative difference between near and far scores, or other quantifiers of spatial trend, stratified by nuclei score range.

The risk assessment sub-process 420 is substantially similar to the risk assessment sub-process 209, except that step 236 is replaced with estimating 440 cancer risks for the patient by applying, to a risk model (e.g., produced via the risk model building sub-process 415), at least some of the set of aggregate scores that are stratified by spatial distribution (e.g., to other cell types) or derivative metrics stratified by score group. For example, the output analytics module 160 may estimate cancer risks by comparing the aggregate scores of particular cell type and spatial distributions to the risk model (e.g., a database of scored samples). The estimate may be performed similarly to the process described above with reference to step 236 in FIG. 2, but may employ a function that is affected by cell-type specific spatial distribution of senescence.

The output analytics module 160 may provide the estimated cancer risks (e.g., output of step 440) for presentation as described above with reference to FIGS. 1 and 2. Note that the process 400 addresses health outcomes related to differences in spatial distributions (clustering of high scores or not, larger vs smaller clusters, often surrounding higher scored nuclei). This can be seen with aggregated scores by distances from high scoring nuclei, or through derivative metrics like half-life and percent difference.

FIG. 5 is a flowchart illustrating a process 500 for predicting cancer risk from direct training on healthy tissue, according to one or more embodiments. The process 500 includes several sub-processes including a prediction model(s) training sub-process 505, an application of prediction model(s) sub-process 510, a risk model building sub-process 515, and a risk assessment sub-process 520. The process and/or sub-processes shown in FIG. 5 may be performed by components of the health prediction computing system 100. Other entities may perform some of the steps in FIG. 5 in other embodiments. Embodiments may include different and/or additional steps, or perform the steps in different orders.

The prediction model(s) training sub-process 505 is substantially similar to the prediction model(s) training sub-process 202, except that step 210 is replaced with step 525 and step 212 is replaced with step 530. The training module 130 may identify 525 nuclei of cells from the one or more training images. For example, the training module 130 may apply segmentation to the images to identify nuclei of some or all of cells present in the images. This segmentation may be performed using techniques such as a U-Net model trained on different cell types (e.g., adipose, stroma, epithelium, etc.) to identify nuclei in the one or more images.

The training module 130 may train 530 one or more cancer risk prediction models (prediction models). For example, the training module 130 can train the one or more prediction models to map images of nuclei (determined in step 525) to health outcome (e.g., develops cancer or not) using classifiers based on numerical score (such as softmax) instead of a senescence state (e.g., like in FIG. 2). Furthermore, instead of expressly extracting a morphology of the nuclei from the segmentation model like in FIG. 2, the training module 130 may train the one or more prediction models directly on raw or normalized images of nuclei that may include non-morphology details, such as details of the nuclei, details of nearby cells and tissue, and may consider nuclear size.

The application of prediction model(s) sub-process 510 is similar to the application of prediction model(s) sub-process 204, except that the prediction module 140 may predict 535 one or more sets of health outcome scores using the one or more prediction models that are associated with different cell types without having to expressly determine cell morphology (e.g., step 220 in FIG. 2). Note that the scores mentioned in step 540 (as part of the application of prediction model(s) sub-process 510) are aggregated health outcome scores (v. aggregated senescence scores in FIG. 2), and the step otherwise operates in a substantially similar manner to the corresponding step described above with reference to FIG. 2. For example, a set of aggregated health outcome scores for various cell types may be the probability of developing cancer or other numerical scores indicating relative likelihood of developing cancer or related conditions.

The risk model building sub-process 515 is similar to the risk model building sub-process 206, except that the risk model builder 150 evaluates 545 biological (e.g., tissue) samples for individuals in a risk training cohort and predicts health outcome scores (v. senescence scores in FIG. 2) using the one or more predictor model(s) trained as part of the prediction model(s) training sub-process 505. And the risk model builder 150 builds 550 the risk model in a similar way as described above with reference to step 232 of FIG. 2, except that the scores are now health outcome scores.

The output analytics module 160 may provide the estimated cancer risks (e.g., output of step 555) for presentation as described above with reference to FIGS. 1 and 2.

Likewise, the risk assessment sub-process 520 is substantially similar to the risk assessment sub-process 209, except step 236 is modified to be step 555 that uses the aggregated health outcome scores of step 540 (v. aggregated senescence scores in FIG. 2).

FIG. 6 is a flowchart illustrating a process 600 for predicting cancer risk from direct training on healthy tissue by morphology, according to one or more embodiments. The process 600 includes several sub-processes including a prediction model(s) training sub-process 605, an application of prediction model(s) sub-process 610, a risk model building sub-process 615, and a risk assessment sub-process 620. The process and/or sub-processes shown in FIG. 6 may be performed by components of the health prediction computing system 100. Other entities may perform some of the steps in FIG. 6 in other embodiments. Embodiments may include different and/or additional steps, or perform the steps in different orders.

The prediction model(s) training sub-process 605 is substantially similar to the prediction model(s) training sub-process 202, except that the training module 130 may train 625 one or more cancer risk prediction models (prediction models). For example, the training module 130 may train one or more machine learning models (i.e., the one or more prediction models) to map images of nuclear morphology (i.e., the determine morphology at step 210) to health outcome (e.g., develops cancer or not), using classifiers based on numerical score (such as softmax). This step may be similar to the analogous step 212 in FIG. 2 but the one or more prediction models map the images of nuclei morphology to health outcomes instead of to senescence state.

The application of prediction model(s) sub-process 610 is similar to the application of prediction model(s) sub-process 204, except that the prediction module 140 may predict 630 the one or more sets of health outcome scores. Note that the scores mentioned in step 635 (as part of the application of prediction model(s) sub-process 610) are aggregated health outcome scores (v. aggregated senescence scores in FIG. 2), and the step otherwise operates in a substantially similar manner to the corresponding step described above with reference to FIG. 2.

The risk model building sub-process 615 is similar to the risk model building sub-process 206, except that the risk model builder 150 evaluates 640 biological (e.g., tissue) samples for individuals in a risk training cohort and predicts health outcome scores (v. senescence scores in FIG. 2) using the one or more predictor model(s) trained as part of the prediction model(s) training sub-process 605. And the risk model builder 150 builds 645 the risk model in a similar way as described above with reference to step 232 of FIG. 2, except that the scores are now health outcome scores.

The output analytics module 160 may provide the estimated cancer risks (e.g., output from step 650) for presentation as described above with reference to FIGS. 1 and 2.

Likewise, the risk assessment sub-process 620 is substantially similar to the risk assessment sub-process 209, except step 236 is modified to be step 650 that uses the aggregated health outcome scores of step 635 (v. aggregated senescence scores in FIG. 2)

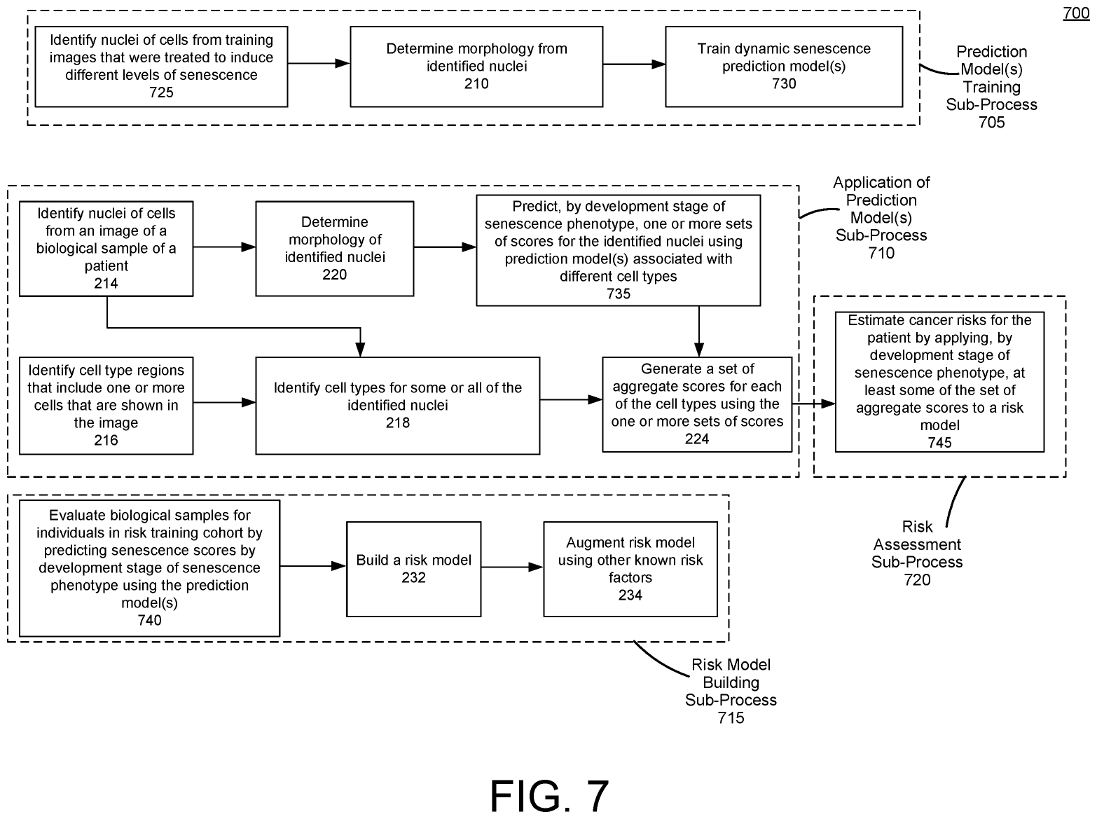

FIG. 7 is a flowchart illustrating a process 700 for predicting cancer risk from developing senescence phenotype, according to one or more embodiments. The process 700 includes several sub-processes including a prediction model(s) training sub-process 705, an application of prediction model(s) sub-process 710, a risk model building sub-process 715, and a risk assessment sub-process 720. The process and/or sub-processes shown in FIG. 7 may be performed by components of the health prediction computing system 100. Other entities may perform some of the steps in FIG. 7 in other embodiments. Embodiments may include different and/or additional steps, or perform the steps in different orders.

The prediction model(s) training sub-process 705 is similar to the prediction model(s) training sub-process 202, except the training module 130 may identify 725 nuclei of cells from training images, where the cells in the training images were treated to induce different stages of senescence. For example, the training module 130 may train one or more machine learning model (i.e., the one or more prediction models) using images of cells in which at least some of the cells were treated to induce replicative senescence by serial passaging or through drug treatment. Where serial passaging is employed, the training images may be captured at multiple time points to capture images of the cells at different stages of senescence phenotype. In some embodiments, the training module 130 may train one or more machine learning model (i.e., the one or more prediction models) using images of cells in which at least some of the cells were treated using a multi-dose drug treatment to induce different levels of development of senescence phenotype. Where multi-dose drug treatment is employed, the training images may be captured with multiple dose levels to achieve different stages of development of senescence phenotype.

In some embodiments, the training module 130 may train 730 one or more prediction models (e.g., dynamic senescence prediction models) using morphology determined at step 210. This approach thus trains the one or more prediction models on images representing the senescent phenotype in different stages of development and/or in different forms reflecting multiple levels of intensity. The training module 130 may train the one or more prediction models to map the determined morphology of nuclei (e.g., images of nuclear morphology) to senescent state or senescence induction dose or day per method (where method refers to how the senescence was induced in the cells of the training images), using classifiers based on numerical score (such as softmax). In some embodiments, the training may be similar to the training described at step 212 of FIG. 2, but instead of mapping images directly to a senescent state, the prediction models are trained to map the images to overall senescence treatment, treatment dose, the passage number, day for replicative senescence, or some combination thereof.

The application of prediction model(s) sub-process 710 is similar to the application of prediction model(s) sub-process 204, except that the prediction module 140 predicts 735, by development stage of senescence phenotype, one or more sets of scores for the identified nuclei using the one or more prediction models that are associated with the different cell types. For example a predicted sets of scores may be for a single nuclei as 0.75 for “early replicative senescence model,” 0.88 for “late replicative senescence model,” 0.31 for “low-dose doxorubicin treatment model.”

The risk model building sub-process 715 is similar to the risk model building sub-process 206, except that the risk model builder 150 evaluates 740 biological (e.g., tissue) samples for individuals in a risk training cohort by predicting senescence scores by development stage of senescence phenotype (v. the more binary senescence scores in FIG. 2) using the one or more predictor model(s) trained as part of the prediction model(s) training sub-process 705.

The risk assessment sub-process 720 is substantially similar to the risk assessment sub-process 209, except step 236 is modified to estimate 745 cancer risks for the patient by applying, by development stage of senescence phenotype, at least some of the set of aggregate scores to a risk model. In some embodiments, the risk model may combine multiple senescence scores, by development stage of senescence phenotype, with external risk factors to estimate the cancers risks.

The output analytics module 160 may provide the estimated cancer risks (e.g., the output of step 745) for presentation as described above with reference to FIGS. 1 and 2.

The process 700 may furthermore alternatively or additionally employ the techniques described in FIG. 2 and/or FIG. 4 in the application of prediction model(s) sub-process 710 and the risk assessment sub-process 720. For example, when generating the aggregate scores for a biological (e.g., tissue) sample, the prediction module 140 may evaluate a distance between cell types and stratify the aggregated scores by distance (as discussed with respect to FIG. 3). Furthermore, the prediction module 140 may optionally identify spatial distributions (e.g., clusters) of nuclei by cell type and score and incorporate this information into the aggregate scores (as discussed with respect to FIG. 4).

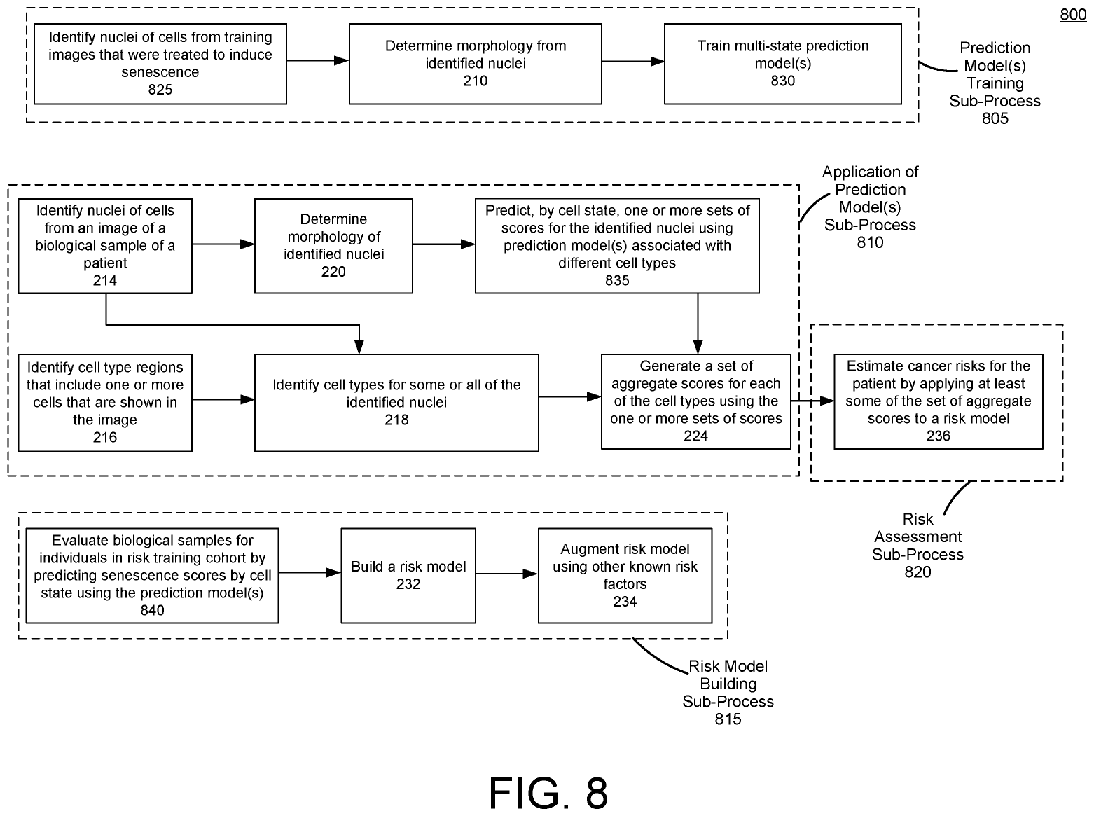

FIG. 8 is a flowchart illustrating a process 800 for predicting cancer risk from multiple cell state phenotype, according to one or more embodiments. The process 800 includes several sub-processes including a prediction model(s) training sub-process 805, an application of prediction model(s) sub-process 810, a risk model building sub-process 815, and a risk assessment sub-process 820. The process and/or sub-processes shown in FIG. 8 may be performed by components of the health prediction computing system 100. Other entities may perform some of the steps in FIG. 8 in other embodiments. Embodiments may include different and/or additional steps, or perform the steps in different orders.

The prediction model(s) training sub-process 805 is similar to the prediction model(s) training sub-process 202, except the training module 130 may identify 825 nuclei of cells from training images, where the cells in the training images were treated to induce a stressed cell state. A stressed cell state may include, e.g., senescence, quiescence, some other stressed cell state, or some combination thereof. For example, multiple cell states may be induced in the cells, such as senescence and quiescence, utilizing one or more methods such as serum starvation and contact inhibition, and other cell stress induced by drug treatments.

In some embodiments, the training module 130 may train 830 one or more prediction models (e.g., multi-state prediction models) using morphology determined at step 210. This approach thus trains the one or more prediction models to map images of nuclear morphology to cell state per method, using classifiers based on numerical score (such as softmax). Here, the one or more prediction models may map the images to experimental conditions such as multiple cell states, treatments, or cell stress inducers.

The application of prediction model(s) sub-process 810 is similar to the application of prediction model(s) sub-process 204, except that the prediction module 140 predicts 835, by cell state, one or more sets of scores for the identified nuclei using the one or more prediction models that are associated with the different cell types. For example, a predicted sets of scores may be for a given nuclei as 0.25 from the “serum starvation quiescence model” and 0.88 from the “chemotherapeutic X stress induced model.”

The risk model building sub-process 815 is similar to the risk model building sub-process 206, except that the risk model builder 150 evaluates 840 biological (e.g., tissue) samples for individuals in a risk training cohort, and predicts senescence scores by cell state (v. the more binary senescence scores in FIG. 2) using the one or more predictor model(s) trained as part of the prediction model(s) training sub-process 805.

The risk assessment sub-process 820 is similar to the risk assessment sub-process 209. In some embodiments, the risk model may combine multiple cell state scores with external risk factors to estimate cancer risks.

The output analytics module 160 may provide the estimated cancer risks (e.g., the output of step 236) for presentation as described above with reference to FIGS. 1 and 2.

As with the process 700 of FIG. 7, the process 800 of FIG. 8 may furthermore alternatively or additionally employ the techniques described in FIG. 3 and/or FIG. 4 in the prediction process such as stratifying the aggregated scores by distance (as discussed with respect to FIG. 3) and aggregating scores based on identified clusters or spatial distribution of nuclei (as discussed with respect to FIG. 4).

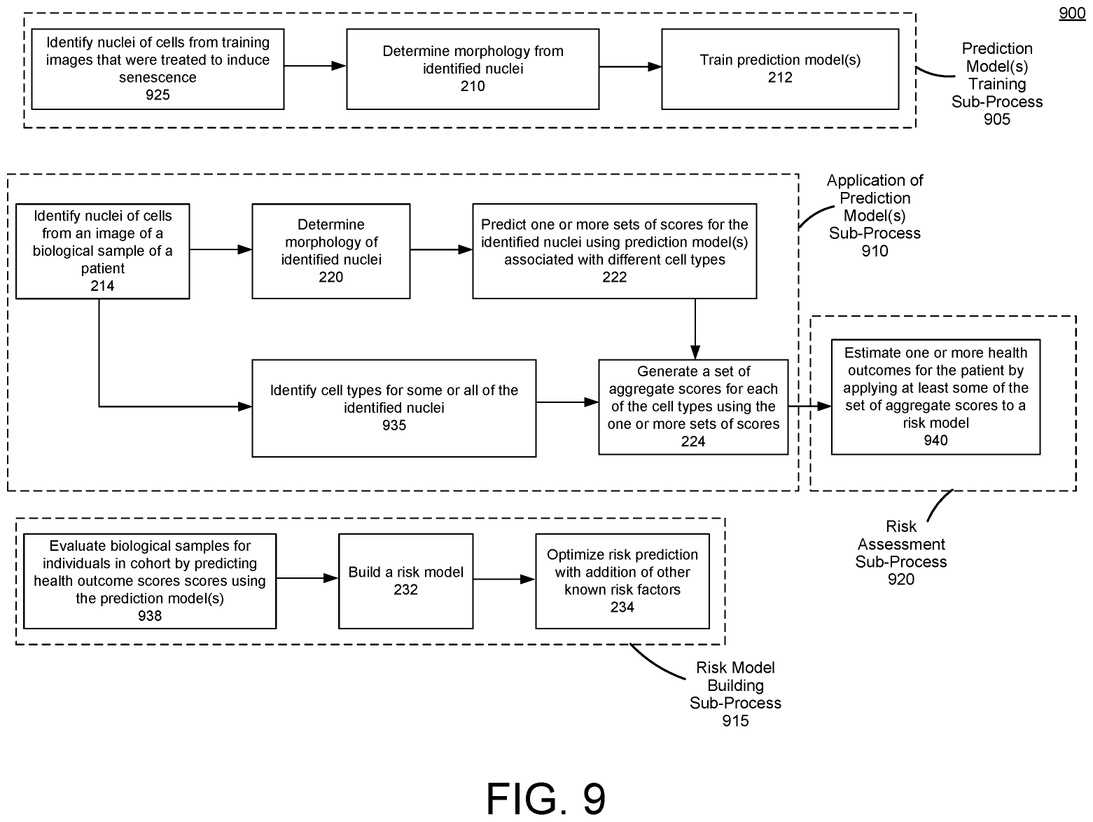

FIG. 9 is a flowchart illustrating a process 900 for predicting a process for predicting inflammation and health outcomes from senescence, according to one or more embodiments. The process 900 includes several sub-processes including a prediction model(s) training sub-process 905, an application of prediction model(s) sub-process 910, a risk model building sub-process 915, and a risk assessment sub-process 920. The process and/or sub-processes shown in FIG. 9 may be performed by components of the health prediction computing system 100. Other entities may perform some of the steps in FIG. 9 in other embodiments. Embodiments may include different and/or additional steps, or perform the steps in different orders.

The prediction model(s) training sub-process 905 is similar to the prediction model(s) training sub-process 202, except the training module 130 may identify 925 nuclei of cells from training images of biological samples (e.g., biofluids), where the cells in the training images were treated to induce senescence. For example, some white blood cells (WBCs) or other cell types (such as fibroblasts) were treated to induce senescence and imaged. The training module 130 then proceeds similarly to the prediction model(s) training sub-process 202 in FIG. 2 to generate the one or more prediction models.

The application of prediction model(s) sub-process 910 is similar to the application of prediction model(s) sub-process 204, however, the biological sample may be a biofluid or other cell sample from the patient. For example, the biofluid may be blood, sputum, or other biofluids that contain white blood cells, such as monocytes or lymphocytes. Another difference between the two sub-processes is that in the sub-process 910, after step 214, the prediction module 140 identifies 935 a cell type of each nucleus of the identified nuclei. Accordingly, one difference is that cell type in a biofluid may be identified individually after identifying nuclei instead of by tissue morphology which can be in parallel. Note that in other embodiments the sub-process 910 may use, e.g., a segmentation model that is done in parallel, independent of identified nuclei for morphological analysis.

The risk model building sub-process 915 is similar to the risk model building sub-process 206, except that the risk model builder 150 evaluates 938 biological (e.g., biofluid) samples for individuals in a risk training cohort by predicting senescence scores using the one or more predictor model(s) trained as part of the prediction model(s) training sub-process 905.

The risk assessment sub-process 920 is similar to the risk assessment sub-process 209. The output analytics module 160 may estimate 940 one or more health outcomes for the patient by applying at least some of the set of aggregate scores to a risk model (generated by the risk model building sub-process 915). Here the health outcomes may include disease prognosis or other outcomes related to immune system function, inflammation and specific diseases such as diabetes and cardiovascular disease. The aggregate score may also indicate the rate of aging.

The output analytics module 160 may provide the estimated one or more health outcomes (e.g., output of step 940) for presentation as described above with reference to FIGS. 1 and 2.

Various aspects of the above-described techniques may be combined in different ways to yield yet further variations of prediction methods consistent with the disclosed embodiments. For example, any of the techniques described in FIGS. 2-8 may be employed to generate risk of inflammation or other health outcomes beyond cancer as shown in the example of FIG. 9. The processes may variously be based on models trained to directly infer senescence (e.g., as shown in FIGS. 2-4, FIG. 7, FIG. 9) or to infer other types of relevant characteristics (e.g., as shown in FIG. 5, FIG. 6, and FIG. 8). Moreover, any of the various techniques for segmenting images and/or performing other pre-processing of the images may be utilized in association with the different types of predictive models described herein.

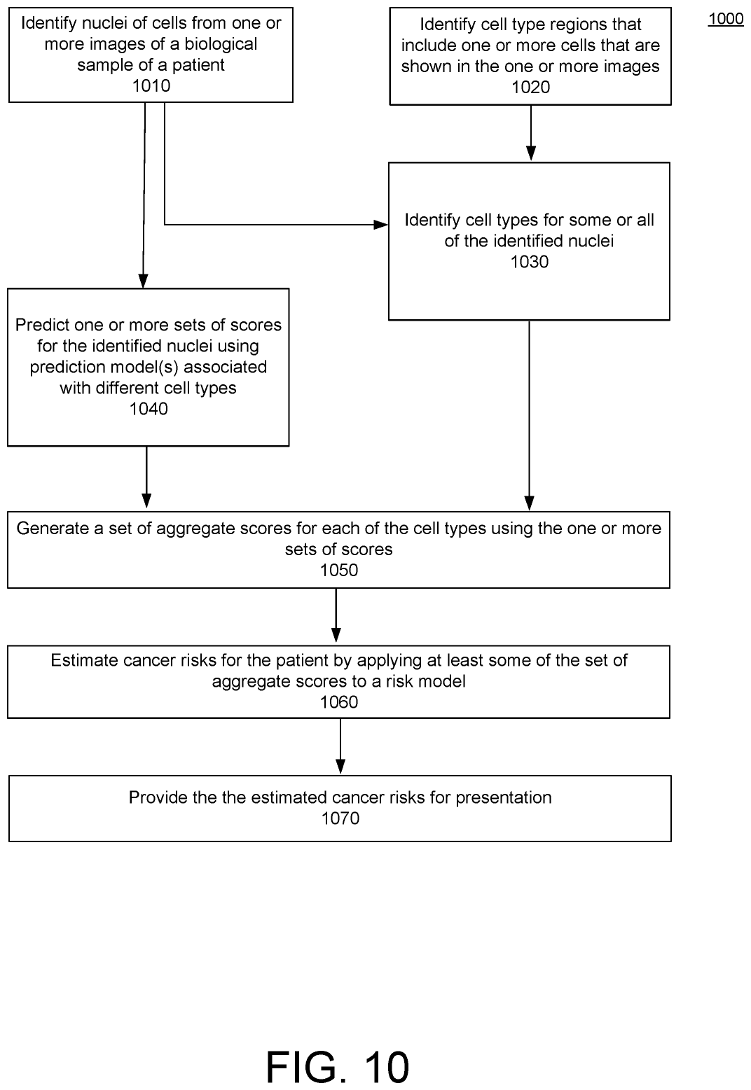

FIG. 10 is a flowchart illustrating a process 1000 for predicting a health condition using one or more prediction models, in accordance with one or more embodiments. A health condition may be, e.g., cancer or a health outcome (e.g., inflammation, disease, etc.). The process shown in FIG. 10 may be performed by components of a health prediction computing system (e.g., the health prediction computing system 100). Other entities may perform some of the steps in FIG. 10 in other embodiments. Embodiments may include different and/or additional steps, or perform the steps in different orders. For example, process 1000 may be modified to perform any of the various application of model(s) sub-processes described above with regard to FIGS. 2-9.

The health prediction computing system identifies 1010 nuclei of cells from one or more images of a biological sample (e.g., tissue) of a patient. For example, a prediction module of the health prediction computing system may apply one or more segmentation techniques to the one or more images to identify nuclei of some or all of cells present in the images. The one or more segmentation techniques may be performed using techniques such as a U-Net model trained on different cell types (e.g., adipose, stroma, epithelium, etc.) to identify nuclei in the one or more images.

The health prediction computing system identifies 1020 cell type regions that include one or more cells that are shown in the one or more images. For example, the prediction module may apply one or more segmentation techniques to the one or more images to identify different cell type regions present in the one or more images. The one or more segmentation techniques may be performed using techniques such as a U-Net model trained with different cell types to predict cell type regions of each image.

The health prediction computing system identifies 1030 cell types for some or all of the identified nuclei. In some embodiments, the health prediction computing system may identify a cell type of each nucleus, of the identified nuclei, based in part on a location of the nucleus relative to some or all of the identified cell type regions. For example, the prediction module may identify a cell type for each nucleus based on detected location of the nucleus relative to the segmented areas of different cells type regions identified in the one or more images. For some (or in some cases all) of the nuclei identified in the one or more images, the prediction module may determine whether the location of the nucleus is associated with an identified cell type based on its location. In some embodiments, the prediction module may map the location of each nucleus to the detected cell type regions to determine overlap.

The health prediction computing system predicts 1040 one or more sets of scores for the identified nuclei using one or more prediction models that are associated with different cell types. The sets of scores may include a respective set of scores for each of the different cell types. The one or more prediction models may be any of the prediction models described above with reference to FIGS. 2-8.

The health prediction computing system generates 1050 a set of aggregate scores for each of the cell types using the sets of scores. The set of aggregate scores may include an aggregate score for each of the different cell types. For example, if there are four different cell types, there would be four aggregate scores, one for each cell type, that together form the set of aggregate scores.

The health prediction computing system estimates 1060 cancer risks for the patient by applying at least some of the set of aggregate scores to a risk model. The risk model may be any of the risk models described above with reference to FIGS. 2-8.

The health prediction computing system provides 1070 the estimated cancer risks for presentation. In some embodiments, the health prediction computing system may provide the estimated cancer risks to a monitor for display. In other embodiments, the health prediction computing system may provide the estimated cancer risks to a client device for display, where the client device is communicatively coupled to the health prediction computing system. In other embodiments, the health prediction computing system may utilize the estimated cancer risk to suggest specific treatments or actions to the patient or care provider on a client device for display or by electronic communications such as email, messages, notifications, etc., or stored for future use to generate reports concerning patients' health.

### Additional Configuration Information

The foregoing description of the embodiments has been presented for illustration; it is not intended to be exhaustive or to limit the patent rights to the precise forms disclosed. Persons skilled in the relevant art can appreciate that many modifications and variations are possible considering the above disclosure.

Some portions of this description describe the embodiments in terms of algorithms and symbolic representations of operations on information. These algorithmic descriptions and representations are commonly used by those skilled in the data processing arts to convey the substance of their work effectively to others skilled in the art. These operations, while described functionally, computationally, or logically, are understood to be implemented by computer programs or equivalent electrical circuits, microcode, or the like. Furthermore, it has also proven convenient at times, to refer to these arrangements of operations as modules, without loss of generality. The described operations and their associated modules may be embodied in software, firmware, hardware, or any combinations thereof.

Any of the steps, operations, or processes described herein may be performed or implemented with one or more hardware or software modules, alone or in combination with other devices. In one embodiment, a software module is implemented with a computer program product comprising a computer-readable medium containing computer program code, which can be executed by a computer processor for performing any or all the steps, operations, or processes described.

Embodiments may also relate to an apparatus for performing the operations herein. This apparatus may be specially constructed for the required purposes, and/or it may comprise a general-purpose computing device selectively activated or reconfigured by a computer program stored in the computer. Such a computer program may be stored in a non-transitory, tangible computer readable storage medium, or any type of media suitable for storing electronic instructions, which may be coupled to a computer system bus. Furthermore, any computing systems referred to in the specification may include a single processor or may be architectures employing multiple processor designs for increased computing capability.

Embodiments may also relate to a product that is produced by a computing process described herein. Such a product may comprise information resulting from a computing process, where the information is stored on a non-transitory, tangible computer readable storage medium and may include any embodiment of a computer program product or other data combination described herein.

Finally, the language used in the specification has been principally selected for readability and instructional purposes, and it may not have been selected to delineate or circumscribe the patent rights. It is therefore intended that the scope of the patent rights be limited not by this detailed description, but rather by any claims that issue on an application based hereon. Accordingly, the disclosure of the embodiments is intended to be illustrative, but not limiting, of the scope of the patent rights, which is set forth in the following claims.

What is claimed is:

1. A method, performed at a computer system comprising a processor and a non-transitory computer readable medium, comprising:
identifying nuclei of cells from an image of a biological sample of a patient;
identifying cell type regions that include one or more cells that are shown in the image;
identifying cell types of the identified nuclei, based in part on locations of the nuclei relative to some or all of the identified cell type regions;
predicting sets of scores for the identified nuclei using prediction models associated with different cell types, and that the sets of scores includes a respective set of scores for each of the different cell types;
generating a set of aggregate scores for each of the cell types using the sets of scores, wherein the set of aggregate scores includes an aggregate score for each of the different cell types; and
estimating a cancer risk for the patient by applying at least some of the set of aggregate scores to a risk model.

2. The method of claim 1, wherein the prediction models are trained to score images of nuclear morphology where the score is representative of a predicted senescent state, the method further comprising:
determining morphology of the identified nuclei,
wherein predicting the sets of scores for the identified nuclei using the prediction models associated with the different cell types comprises:
applying the determined morphology of the identified nuclei to the prediction models to obtain the sets of scores.

3. The method of claim 2, wherein the prediction models are dynamic senescence predictor models that were trained using images of cells in which at least some of the cells were treated to induce a range of development stages of senescence phenotype, and the predicted sets of scores are associated with different development stages of senescence phenotype.

4. The method of claim 2, wherein the prediction models are multi-state predictor models that were trained using images of cells in which at least some of the cells were treated to induce one or more stressed cell states, and the predicted sets of scores are associated with one or more cell states.

5. The method of claim 2, further comprising:
determining distances between the identified cell types; and
wherein generating the set of aggregate scores for each of the cell types using the sets of scores, further comprises:
stratifying the sets of scores by the determined distances,
wherein estimating the cancer risks for the patient by applying at least some of the set of aggregate scores to the risk model, further comprises:
comparing the aggregate scores for each cell type, stratified by distance to other cell types to a database of scored samples.

6. The method of claim 2, further comprising:
determining distances between the identified nuclei, wherein the scores are stratified by distance to other score-stratified nuclei, from the predicted sets of scores;
determining spatial distributions from identified nuclei based in part on their score from the predicted sets of scores and cell type; and
generating derivative metrics that characterize the spatial distributions of scores relative to nuclei stratified by score range;
wherein generating the set of aggregate scores for each of the cell types using the sets of scores, further comprises:
generating a set of aggregate scores that are stratified by spatial distribution or derivative metrics that are stratified by score groups for each cell type.

7. The method of claim 1, wherein the prediction models are trained to predict health outcome scores using nuclear morphology, and the sets of scores are sets of health outcome scores, the method further comprising:
determining morphology of the identified nuclei,
wherein predicting the sets of scores for the identified nuclei using the prediction models associated with the different cell types comprises:
applying the determined morphology of the identified nuclei to the prediction models to obtain the sets of health outcome scores.

8. The method of claim 1, wherein the prediction models are trained to predict health outcomes using images of nuclei, and predicting the sets of scores for the identified nuclei using the prediction models associated with the different cell types comprises:
applying the identified nuclei to the prediction models to obtain the sets of scores.

9. A method, performed at a computer system comprising a processor and a non-transitory computer readable medium, comprising:
identifying nuclei of cells from an image of a biological sample of a patient;
identifying a cell type of each nucleus of the identified nuclei;
determining morphology of the identified nuclei;
predicting sets of scores for the identified nuclei using prediction models associated with different cell types, and that the sets of scores includes a respective set of scores for each of the different cell types;
generating a set of aggregate scores for each of the cell types using the sets of scores, wherein the set of aggregate scores includes an aggregate score for each of the different cell types; and
estimating a health outcome for the patient by applying at least some of the set of aggregate scores to a risk model.

10. The method of claim 9, wherein the biological sample is a biofluid.

11. The method of claim 9, wherein identifying the cell type of each nucleus of the identified nuclei is performed in parallel with determining the morphology of the identified nuclei.

12. A non-transitory computer-readable storage medium comprising stored instructions, the instructions when executed by a processor of a device, causing the device to:
identify nuclei of cells from an image of a biological sample of a patient;
identify cell type regions that include one or more cells that are shown in the image;
identify cell types of the identified nuclei, based in part on locations of the nuclei relative to some or all of the identified cell type regions;
predict sets of scores for the identified nuclei using prediction models associated with different cell types, and that the sets of scores includes a respective set of scores for each of the different cell types;
generate a set of aggregate scores for each of the cell types using the sets of scores, wherein the set of aggregate scores includes an aggregate score for each of the different cell types; and
estimate cancer risks for the patient by applying at least some of the set of aggregate scores to a risk model.

13. The non-transitory computer-readable storage medium of claim 12, wherein the prediction models are trained to score images of nuclear morphology where the score is representative of a predicted senescent state, further comprising stored instructions that when executed cause the device to:
determine morphology of the identified nuclei,
wherein the stored instructions to predict the sets of scores for the identified nuclei using the prediction models associated with the different cell types further comprises stored instruction that when executed cause the device to:
apply the determined morphology of the identified nuclei to the prediction models to obtain the sets of scores.

14. The non-transitory computer-readable storage medium of claim 13, wherein the prediction models are dynamic senescence predictor models that were trained using images of cells in which at least some of the cells were treated to induce a range of development stages of senescence phenotype, and the predicted sets of scores are associated with different development stages of senescence phenotype.

15. The non-transitory computer-readable storage medium of claim 13, wherein the prediction models are multi-state predictor models that were trained using images of cells in which at least some of the cells were treated to induce one or more stressed cell states, and the predicted sets of scores are associated with one or more cell states.

16. The non-transitory computer-readable storage medium of claim 13, further comprising stored instructions that when executed cause the device to:
determine distances between the identified cell types; and
wherein the stored instructions to generate the set of aggregate scores for each of the cell types using the sets of scores, further comprises stored instruction that when executed cause the device to:
stratify the sets of scores by the determined distances,
wherein the stored instructions to estimate the cancer risks for the patient by applying at least some of the set of aggregate scores to the risk model, further comprises stored instruction that when executed cause the device to:
compare the aggregate scores for each cell type, stratified by distance to other cell types to a database of scored samples.

17. The non-transitory computer-readable storage medium of claim 13, further comprising stored instructions that when executed cause the device to:
determine distances between the identified nuclei, wherein the scores are stratified by distance to other score-stratified nuclei, from the predicted sets of scores;
determine spatial distributions from identified nuclei based in part on their score from the predicted sets of scores and cell type; and
generate derivative metrics that characterize the spatial distributions of scores relative to nuclei stratified by score range;
wherein the stored instructions to generate the set of aggregate scores for each of the cell types using the sets of scores, further comprises stored instruction that when executed cause the device to:
generate a set of aggregate scores that are stratified by spatial distribution or derivative metrics that are stratified by score groups for each cell type.

18. The non-transitory computer-readable storage medium of claim 12, wherein the prediction models are trained to predict health outcome scores using nuclear morphology, and the sets of scores are sets of health outcome scores, and the non-transitory computer-readable storage medium further comprising stored instructions that when executed cause the device to:
determine morphology of the identified nuclei,
wherein the stored instructions to predict the sets of scores for the identified nuclei using the prediction models associated with the different cell types, further comprises stored instruction that when executed cause the device to:
apply the determined morphology of the identified nuclei to the prediction models to obtain the sets of health outcome scores.

19. The non-transitory computer-readable storage medium of claim 12, wherein the prediction models are trained to predict health outcomes using images of nuclei, and where the stored instructions to predict the sets of scores for the identified nuclei using the prediction models associated with the different cell type, further comprises stored instruction that when executed cause the device to:
apply the identified nuclei to the prediction models to obtain the sets of scores.

20. The non-transitory computer-readable storage medium of claim 12, wherein the biological sample is a sample of tissue.
